(19) United States
(12) Patent Application Publication
(10) Pub. No.: US 2017/0153222 A1
(43) Pub. Date: Jun. 1, 2017

(54) BLOOD COAGULATION TESTING APPARATUS

(71) Applicant: GWANGJU INSTITUTE OF SCIENCE AND TECHNOLOGY, Gwangju (KR)

(72) Inventors: Jong-Hyun LEE, Gwangju (KR); Hyeon-Woo KIM, Gwangju (KR); Chang-Ju PARK, Gwangju (KR); Seung-Wan SEO, Gwangju (KR)

(21) Appl. No.: 15/365,839

(22) Filed: Nov. 30, 2016

(30) Foreign Application Priority Data
Dec. 1, 2015 (KR) 10-2015-0170184

(51) Int. Cl. G01N 33/49

(52) U.S. Cl. CPC G01N 33/4905

(57) ABSTRACT

A blood coagulation testing apparatus for determining compatibility between donor's blood and patient's blood before blood transfusion. The blood coagulation testing apparatus includes: a cylindrical main body connected at one end thereof to a supply tube for delivering donor's blood and connected at the other end thereof to a blood transfer tube for introduction of patient's blood, wherein the main body is divided into two sections by a barrier film extending in a longitudinal direction of the main body such that blood is introduced into the main body through two paths, wherein the donor's blood and the patient's blood are introduced into one of the two sections through one end of the main body and the other end of the main body, respectively, and wherein the barrier film is formed on a surface thereof with a blood coagulation indicator for checking coagulation.

### CROSS REFERENCE TO RELATED APPLICATION

This application claims the benefit of Korean Patent Application No. 10-2015-0170184, filed on Dec. 1, 2015, entitled “BLOOD COAGULATION MEASURING DEVICE”, which is hereby incorporated by reference in its entirety into this application.

### BACKGROUND

1. Technical Field

The present invention relates to a blood coagulation testing apparatus, and, more particularly, to a blood coagulation testing apparatus which can determine compatibility between blood for transfusion and blood of a patient before blood transfusion.

2. Description of the Related Art

Blood transfusion is a medical procedure in which blood taken from a healthy person or a component thereof is injected into a blood vessel of a patient. Before blood transfusion, whether donor's blood is compatible with patient's blood must be checked. For this purpose, ABO typing, Rh typing, cross matching, and the like are utilized.

Generally, blood transfusion is composed of procedures including prescription by a doctor, a cross-matching test between donor's blood and recipient's blood, delivery of blood products from a blood bank, recipient identification, and transfusion. Particularly, recipient identification, which is a final step before transfusion, requires the utmost care to prevent transfusion to a wrong recipient that can cause a fatal medical accident. If an incompatible blood type is transfused, it causes coagulation and hemolysis, can also cause hematuria, acute renal failure, algidity, backache, chest pain, headache, nausea, febrility, hypotension, asthenia, and the like, and, at worst, can cause shock or lead to death due to disseminated intravascular coagulation.

Such medical accidents have been reported every year, causing concern to patients and their families. This is mostly due to the fact that, in recipient identification, medical staff relies heavily on identification of information written on a blood pack and patient's demographic information, thereby increasing the risk of transfusion accident. Since such a transfusion accident can occur due to error or carelessness of medical staff regardless of development of medical technology, medical education alone is not enough to prevent the accident. Therefore, there is a need for a means that can technically determine transfusion compatibility so as to prevent transfusion accidents.

### BRIEF SUMMARY

Embodiments of the present invention have been conceived to solve such a problem in the art and it is an aspect of the present invention to provide a blood coagulation testing apparatus which can determine compatibility of donor blood before transfusion, thereby preventing administration of an incompatible blood transfusion due to carelessness of medical staff.

In accordance with an aspect of the present invention, a blood coagulation testing apparatus for determining compatibility between donor's blood and patient's blood before blood transfusion includes: a cylindrical main body connected at one end thereof to a supply tube for delivering donor's blood and connected at the other end thereof to a blood transfer tube for introduction of patient's blood, wherein the main body is divided into two sections by a barrier film extending in a longitudinal direction of the main body such that blood is introduced into the main body through two paths, wherein the donor's blood and the patient's blood are introduced into one of the two sections through one end of the main body and the other end of the main body, respectively, and wherein the barrier film is formed on a surface thereof with a blood coagulation indicator for checking coagulation.

According to the present invention, the blood coagulation testing apparatus can determine compatibility of donor blood before transfusion, thereby preventing administration of an incompatible blood transfusion due to carelessness of medical staff.

According to the present invention, the blood coagulation testing apparatus can be connected to an existing transfusion line in a simple manner to determine transfusion compatibility based on occurrence of coagulation, thereby reducing the risk of a medical accident at low cost while allowing a patient to visually verify compatibility of donor blood, thereby relieving anxiety about blood transfusion.

### BRIEF DESCRIPTION OF THE DRAWINGS

The above and other aspects, features, and advantages of the present invention will become apparent from the detailed description of the following embodiments in conjunction with the accompanying drawings, in which;

FIG. 1 is a sectional view of a blood transfusion apparatus according to one embodiment of the present invention;

FIG. 2 is a view of a blood coagulation testing apparatus according to one embodiment of the invention;

FIG. 3 is a cross-sectional view taken along line A-A′ of FIG. 2;

FIG. 4 is a cross-sectional view taken along line B-B′ of FIG. 2;

FIG. 5 is a view of an outer surface of the blood coagulation testing apparatus according to one embodiment of the invention;

FIG. 6 is a sectional view of a first section of the blood coagulation testing apparatus according to one embodiment of the invention;

FIG. 7 is a sectional view of a blood coagulation testing apparatus according to another embodiment of the invention;

FIG. 8 is a cross-sectional view taken along line C-C′ of FIG. 7; and

FIGS. 9 and 10 are views showing operation of a valve of the blood coagulation testing apparatus according to another embodiment of the invention;

### DETAILED DESCRIPTION

Hereinafter, embodiments of the present invention will be described with reference to the accompanying drawings. It should be noted that like components will be denoted by like reference numerals throughout the specification and the accompanying drawings. In addition, descriptions of details apparent to those skilled in the art will be omitted for clarity.

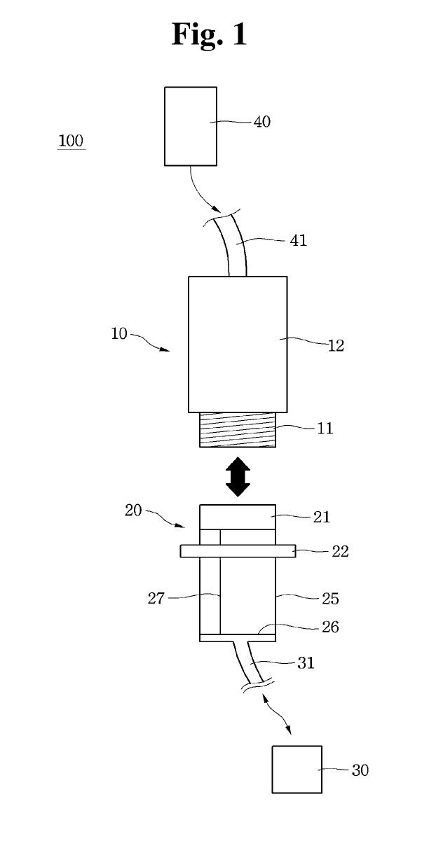

FIG. 1 is a sectional view of a blood transfusion apparatus including a blood coagulation testing apparatus according to one embodiment of the present invention.

Referring to FIG. 1, a blood transfusion apparatus 100 according to this embodiment may include a blood storage unit 40 containing donor's blood, a supply tube 41 providing a path for blood, a coupling unit 10 connected to the supply tube, a blood coagulation testing apparatus 20 fastened to the coupling unit 10, a blood transfer tube 31, and a unit 30 for connection to a blood vessel of a patient.

The blood storage unit 40 stores donor's blood and includes a blood transfusion pack with information on the blood written thereon. The blood in the blood storage unit 40 may be delivered to the coupling unit 10 through the supply tube 41.

The coupling unit 10 may be composed of a cylindrical body 12 and a fastening portion 11 at a lower end of the body. The coupling unit 10 connects the blood coagulation testing apparatus 20 according to this embodiment to the path of blood and serves as a buffer for holding donor's blood before the blood is delivered to the blood coagulation testing apparatus to check coagulation. The fastening portion 11 at the lower end of the coupling unit 10 may have a mating surface having a helical protrusion.

The apparatus 20 according to this embodiment may include a fastening portion 21, a clamp 22, a main body 25, and a barrier film 27. The fastening portion 21 may have a cylindrical shape to be coupled to the fastening portion 11 at the lower end of the coupling unit 12 and have a mating groove formed on an inner surface thereof to be fastened to a threaded surface of the fastening portion 11 by screwing the coupling unit 12.

The main body 25 has a cylindrical shape and is disposed at a lower end of the fastener portion 21, and the barrier film 27 is formed on one side of the main body 25 to divide the main body into two sections. The barrier film 27 has a flat shape and serves to block flow of blood between the sections while dividing the main body 25 into two sections. The main body 25 is provided at a lower end thereof with a membrane 26 which transmits patient's blood delivered from the blood transfer tube 31 into the main body 25.

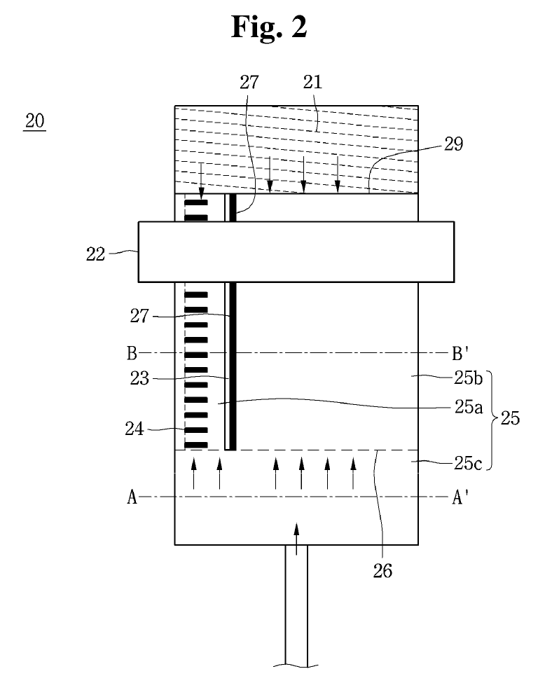

FIG. 2 is a view of a blood coagulation testing apparatus according to one embodiment of the present invention.

A blood coagulation testing apparatus 20 according to this embodiment will now be described in detail with reference to FIG. 2. A main body 25 may have a hollow cylindrical tube shape and be formed of a flexible material to be easily deformed when a portion of the main body 25 is depressed. The main body 25 is provided thereon with a clamp 22. The clamp serves to compress the main body 25 to block flow of blood from the blood transfusion pack into the main body 25.

The main body 25 is provided therein with a barrier film which divides the main body 25 into a first section 25a and a second section 25b. Flow of blood between the first section 25a and the second section 25b is blocked by the barrier film, and members for checking coagulation may be disposed in the first section 25a. A plurality of columns 24 protruding towards the barrier film 27 and having a micro-scale diameter may be formed in the first section 25a, and blood introduced into the first section 25a may flow in a space between the barrier film 27 and the columns 24. In addition, a coagulation indicator 23 may be formed on a surface of the barrier film facing the columns 24.

The barrier film 27 is provided at both ends thereof with blood permeable membranes. Specifically, a first membrane 29 may be placed at one end of the barrier film 27 adjoining the fastening portion 21 and a second membrane 26 may be placed at the other end of the barrier film 27. The first membrane 29 allows blood from the blood transfusion pack to flow into the main body 25 in a direction of upper arrows and the second membrane 26 allows patient's blood to flow into the first section 25a and the second section 25b of the main body 25 in a direction of lower arrows. A third section 25c may be defined by the first membrane 26.

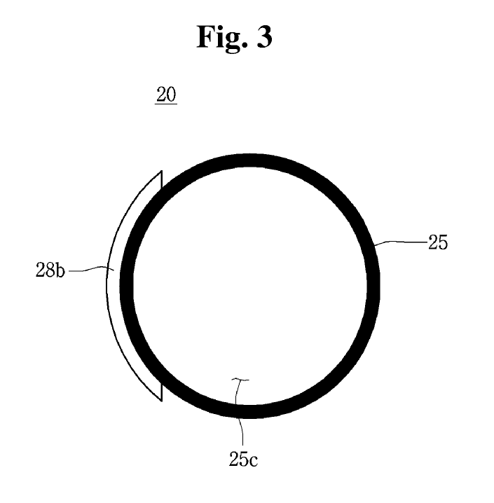

FIG. 3 is a cross-sectional view taken along line A-A′ of FIG. 2.

Specifically, FIG. 3 is a cross-sectional view of the third section 25c of the main body 25 of the blood coagulation testing apparatus 20. Referring to FIG. 3, the main body 25 has a hollow cylindrical tube shape. The third section 25c is a space through which patient's blood flows into the first and second sections of the blood coagulation testing apparatus.

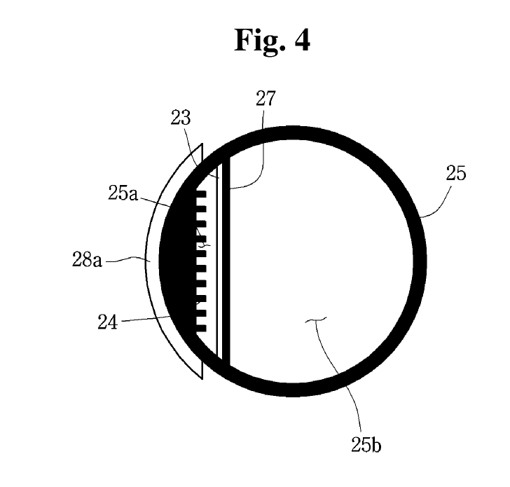

FIG. 4 is a cross-sectional view taken along line B-B′ of FIG. 2.

Specifically, FIG. 4 is a cross-sectional view of the first section 25a and the second section 25b of the main body 25 of the blood coagulation testing apparatus 20. Referring to FIG. 4, the main body 25 is divided into the first section 25a and the second section 25b occupying different areas by the barrier film 27. The first section 25a is a region in which donor's blood is mixed with patient's blood, and may be provided with the coagulation indicator 23 inside the barrier film 27 and be formed on a surface thereof opposite the coagulation indicator with a plurality of protrusions 24. In addition, the main body may be provided on an outer surface thereof corresponding to the first section 25a with a cover 25 surrounding the first section 25a.

The coagulation indicator 23 may be formed of a material capable of absorbing blood, such as paper, and allows blood coagulation to be checked for each blood type (A, B, O, AB, RH⁺, RH⁻) upon mixing of donor's blood with patient's blood.

The protrusions 24 may be an array of columns having a size of tens of micrometers and allow donor's blood to be rapidly mixed with patient's blood, thereby allowing blood coagulation to be checked in a shorter time. The cover 28 on the outer surface of the main body serves to facilitate mixing. Specifically, the cover 28 may be depressed inward such that blood samples in the first section 25a can be mixed by the protrusions 24, thereby allowing blood coagulation to be checked in a shorter time.

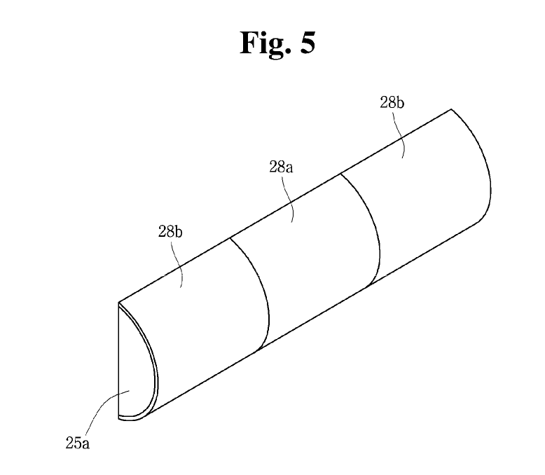

FIG. 5 is a view of an outer surface of the blood coagulation testing apparatus according to the embodiment.

Referring to FIG. 5, the blood coagulation testing apparatus may include covers covering an outer surface of the first section 25a occupying a portion of the main body. Specifically, a first cover 28a formed of a transparent polymer may be disposed in a region of the outer surface in which the protrusions are formed to allow blood samples to be mixed by depressing the main body, as shown in FIG. 4. In addition, a second cover 28b formed of a plastic or lens may be disposed in a region of the outer surface in which the protrusions are not formed.

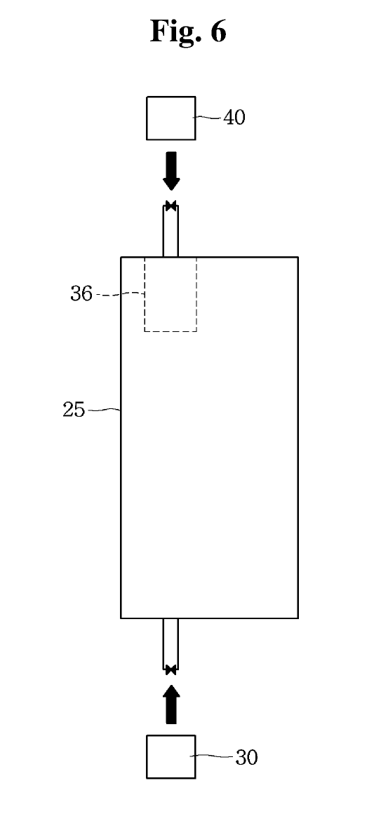

FIG. 6 is a sectional view of the first section of the blood coagulation testing apparatus according to the embodiment. Referring to FIG. 6, the blood coagulation testing apparatus 20 according to the embodiment may include a microfilter 36 connected to a supply tube for delivering donor's blood. The microfilter 36 filters out red blood cells (RBCs) and white blood cells (WBCs) from the donor's blood so as to allow only plasma to pass therethrough, thereby facilitating coagulation.

Preferably, the first section is vacuum-packed to allow donor's blood and patient's blood to easily flow thereinto. Although not shown in the drawings, a vacuum pump for evacuating the first section may be placed on an outer side surface of the main body.

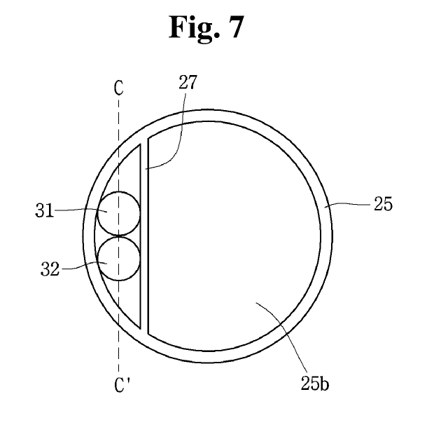

FIG. 7 is a cross-sectional view of a blood coagulation testing apparatus according to another embodiment of the present invention. Specifically, FIG. 7 is a cross-sectional view of a cross section of a first section 25a and a second section 25b of a main body 25 of a blood coagulation testing apparatus according to another embodiment of the invention.

The main body 25 is divided into two sections, i.e. a first section 25a and a second section 25b occupying different areas by a vertical barrier film placed inside the main body. The first section may be provided therein with a pair of receptacles 31, 32 containing different reagents.

Although two receptacles are used to check coagulation in this example, it should be understood that three or more receptacles may be used to conduct coagulation testing on the basis of ABO blood group compatibility.

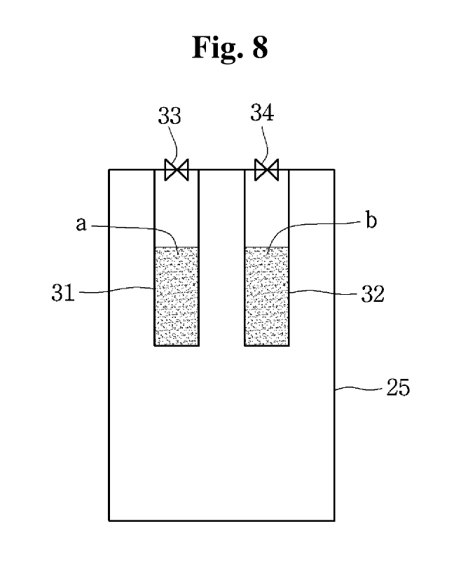

FIG. 8 is a cross-sectional view taken along line C-C′ of FIG. 7.

Referring to FIG. 8, in the blood coagulation testing apparatus according to this embodiment, the main body may be divided into two sections, one of which receives the first receptacle 31 and the second receptacle 32 therein, as shown in FIG. 7. For example, the first receptacle 31 may contain reagent A such as anti-B antibody, and the second receptacle 32 may contain reagent B such as anti-A antibody.

Valves 33, 34 for introduction of patient's blood may be provided at upper ends of the first and second receptacles 31, 32, respectively. When the valves 33, 34 are open, the patient's blood is introduced into the first and second receptacles to react with the reagents, thereby determining compatibility with donor's blood before transfusion.

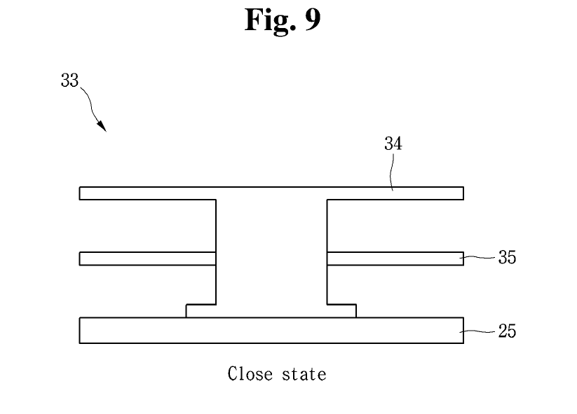

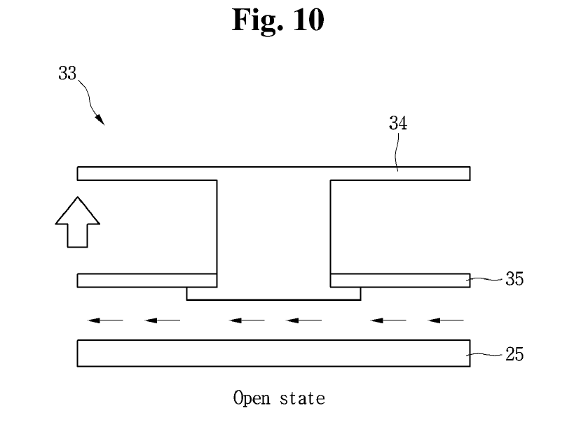

FIGS. 9 and 10 are views showing operation of the valve of the blood coagulation testing apparatus according to this embodiment.

FIG. 9 shows the valve 33 for the first receptacle 31, with the valve 33 in a closed state. The valve 33 may include a T-shaped movable member 34 configured to be brought into contact with the main body 25 and a membrane 35 placed across the movable member 34. The movable member 34 may be composed of an upper end serving as a handle, a body, and a lower end, which may have different widths.

FIG. 10 shows the valve 33 for the first receptacle 31 in an open state. When the movable member 34 is moved upward, the valve is opened. The membrane 35 adjoins the body of the movable member. Since the lower end of the movable member 34 has a larger width than the body of the movable member, upward movement of the lower end of the movable member is stopped by the membrane 35 to create a passage for flow of patient's blood into the receptacle when the movable member 34 is moved upward. In this way, the patient's blood may be introduced into the receptacle containing the reagent to determine compatibility with donor's blood. In other words, the blood coagulation testing apparatus according to this embodiment of the invention can verify the blood type of a patient, thereby finally determining whether the donor's blood is compatible with the patient's blood before transfusion.

Next, a method for determining transfusion compatibility using the blood coagulation testing apparatus according to the present invention will be described.

The blood coagulation testing apparatus according to any one of the embodiments of the invention may be integrally formed with an existing blood vessel connection device. A supply tube for delivering donor's blood may be provided at one end thereof with a connecting portion for fastening of the blood coagulation testing apparatus. The blood coagulation testing apparatus may be fabricated as a disposable diagnostic device coupled to the connecting portion of the supply tube and the blood vessel connection device.

After the blood coagulation testing apparatus is fastened to the connecting portion, donor's blood is introduced into the main body of the blood coagulation testing apparatus through one end of the blood coagulation testing apparatus. The main body is divided into two sections including the first and second sections, and the blood is introduced into both of the sections.

Patient's blood from the blood vessel connection device connected to a patient is introduced through the other end of the blood coagulation testing apparatus. The patient's blood is also introduced into both of the first and second sections.

A donor's blood sample and a patient's blood sample are simultaneously introduced into the first section of the blood coagulation testing apparatus. When the cover on the outer surface of the first section is depressed, the protrusions formed on the first section allow the blood samples to be mixed to promote coagulation. The blood coagulation indicator capable of absorbing the blood samples is provided inside the first section. When antibodies of the blood samples react with one another and agglomerate, the blood coagulation indicator undergoes a certain change, based on which transfusion compatibility can be determined.

If coagulation is detected by the blood coagulation testing apparatus, the clamp is closed to block flow of blood into the blood coagulation apparatus, followed by withdrawal of transfusion subsequent to disconnecting the blood coagulation testing apparatus from the connecting portion. Then, another blood pack may be connected to the blood coagulation testing apparatus to conduct coagulation testing.

If coagulation is not detected, the clamp is opened to provide a path for donor's blood. The donor's blood from the supply tube may be delivered to a patient through the main body.

As described above, the blood coagulation testing apparatus according to the present invention can determine compatibility of donor's blood before transfusion, thereby preventing administration of an incompatible blood transfusion due to carelessness of medical staff.

In addition, the blood coagulation testing apparatus according to the present invention can be connected to an existing transfusion line in a simple manner to determine transfusion compatibility based on occurrence of coagulation, thereby reducing the risk of a medical accident at low cost while allowing a patient to visually verify compatibility of donor's blood, thereby relieving anxiety about blood transfusion.

Although the present invention has been described with reference to some embodiments, it should be understood that these embodiments are provided for illustration only and are not to be construed in any way as limiting the present invention, and that various modifications, changes, alterations, and variations can be made by those skilled in the art without departing from the spirit and scope of the invention. For example, each component described in the embodiments can be modified in various forms. In addition, differences relating to these modifications and applications are to be construed as within the scope of the invention defined in the appended claims.

What is claimed is:

1. A blood coagulation testing apparatus for determining compatibility between donor's blood and patient's blood before blood transfusion, comprising:
a cylindrical main body connected at one end thereof to a supply tube for delivering donor's blood and connected at the other end thereof to a blood transfer tube for introduction of patient's blood,
wherein the main body is divided into two sections by a barrier film extending in a longitudinal direction of the main body such that blood is introduced into the main body through two paths,
wherein the donor's blood and the patient's blood are introduced into one of the two sections through one end of the main body and the other end of the main body, respectively, and
wherein the barrier film is formed on a surface thereof with a blood coagulation indicator for checking coagulation.

2. The blood coagulation testing apparatus according to claim 1, wherein the one of the two sections divided by the barrier film is formed on an inner surface thereof opposite the barrier film with a plurality of protrusions.

3. The blood coagulation testing apparatus according to claim 2, wherein the protrusions are spaced a predetermined distance from the barrier film such that a space for mixing the patient blood with the donor's blood is formed therebetween.

4. The blood coagulation testing apparatus according to claim 1, further comprising:
a coupling unit connected to the supply tube,
the coupling unit being composed of a cylindrical body and a fastening portion for coupling to the main body.

5. The blood coagulation testing apparatus according to claim 4, wherein the fastening portion is fastened to a mating surface provided at one end of the main body.

6. The blood coagulation testing apparatus according to claim 1, wherein the main body is provided at opposite ends thereof facing the supply tube and the blood transfer tube with membranes through which blood is introduced into the main body.

7. The blood coagulation testing apparatus according to claim 6, wherein the barrier film is formed in a direction perpendicular to the membrane to divide the main body into a first section and a second section.

8. The blood coagulation testing apparatus according to claim 7, wherein the first section has a smaller cross-sectional area than the second section.

9. The blood coagulation testing apparatus according to claim 7, further comprising:
a first cover covering a portion of an outer surface of the main body corresponding to the first section,
wherein the first cover is formed of a transparent polymer.

10. The blood coagulation testing apparatus according to claim 7, further comprising:
a second cover covering a portion of the outer surface of the main body corresponding to the second section,
wherein the second cover is formed of a transparent plastic or lens.

11. The blood coagulation testing apparatus according to claim 1, further comprising:
a clamp surrounding the main body and compressing the main body to block flow of blood into the main body.

12. The blood coagulation testing apparatus according to claim 10, wherein, when coagulation does not occur in the main body, the clamp is opened to initiate transfusion, and, when coagulation occurs in the main body, the donor's blood is separated from the patient, with the clamp in a closed state.

13. The blood coagulation testing apparatus according to claim 1, further comprising:
a microfilter provided to the main body to filter out red blood cells and white blood cells from the patient's blood delivered through the blood transfer tube.

14. The blood coagulation testing apparatus according to claim 1, further comprising:
a vacuum pump disposed on a side surface of the main body to evacuate the section comprising the protrusions.

15. A blood coagulation testing apparatus for determining compatibility between donor's blood and patient's blood before blood transfusion, comprising:
a cylindrical main body connected at one end thereof to a supply tube for delivering donor's blood and connected at the other end thereof to a blood transfer tube for introduction of patient's blood,
wherein the main body comprises a barrier film extending in a longitudinal direction of the main body to form two paths for flow of blood into the main body,
wherein the main body is divided into a first section and a second section by the barrier film, the first section being provided therein with a plurality of tubes containing different reagents, and
wherein each of the tubes is provided at one end thereof with a shut-off valve for introduction of the patient's blood.
